(19) United States
(12) Patent Application Publication
(10) Pub. No.: US 2018/0201447 A1
(43) Pub. Date: Jul. 19, 2018

(54) TREATMENT FACILITY COMPRISING A TRANSFER DEVICE BETWEEN A ZONE AT ATMOSPHERIC PRESSURE AND A ZONE UNDER VACUUM, AND CORRESPONDING METHOD OF IMPLEMENTATION

(71) Applicant: COATING PLASMA INDUSTRIE, Peynier (FR)

(72) Inventor: Jocelyn VIARD, Sausset-Les-Pins (FR)

(73) Assignee: COATING PLASMA INDUSTRIE, Peynier (FR)

(21) Appl. No.: 15/742,590

(22) PCT Filed: Jun. 29, 2016
(86) PCT No.: PCT/FR2016/051612

§ 371 Date: Jan. 8, 2018

(30) Foreign Application Priority Data
Jul. 13, 2015 (FR) 15 56641

(51) Int. Cl. B65G 17/06; B65G 29/02; B65G 59/06; A61L 2/26; A61L 2/14

(52) U.S. Cl. CPC B65G 17/063; B65G 29/02; B65G 59/061; A61L 2202/23; A61L 2/14; B65G 2201/0258; A61L 2/26

(57) ABSTRACT

A facility having a main space under vacuum, at least one device for transferring objects between a zone at atmospheric pressure and a zone under vacuum, a device (30) for unstacking/stacking objects, a device for conveying objects and support elements (24, 25) for supporting each object, these being secured to the conveying device. Each transfer device has a frame and a transfer member, provided with at least one cell (C1-C4) that accepts at least one object that is to be transferred. The frame defines a first opening in communication with the zone at atmospheric pressure, a second opening in communication with the zone under vacuum, and an intermediate region intended to be placed either under vacuum or at atmospheric pressure.

### TECHNICAL FIELD OF THE INVENTION

The invention relates to vacuum technology and vacuum treatments. It relates to a vacuum treatment facility provided with at least one device for transferring objects between a zone at atmospheric pressure and a zone under vacuum, as well as to a method for implementing this treatment facility. As a non-limiting example, the objects can be in particular hollow objects, having an interior volume delimited by a wall that has an inner face and an outer face. Such objects are among others goblets, cups, trays, jars, tubes or industrial molds.

The invention relates more particularly to such a facility, wherein the aforementioned objects are introduced into the vacuum treatment enclosure by the transfer device, continuously and at a high rate of speed.

### PRIOR ART

Vacuum plasma treatment methods require that the parts to be treated be placed in a vacuum. In prior art, the setting in place of objects is generally carried out in batches. This is a long process that is not very compatible with high speeds.

Mention will first be made, as a document representing this prior art, of U.S. Pat. No. 8,336,448. After setting the objects in place in the plasma chamber, the latter is placed under vacuum in order to generate a type of plasma for a determined treatment time.

A method is moreover known, wherein the objects to be treated reside in successive plasma chambers. Each chamber is closed by airlocks which make it possible to keep a vacuum pressure in each chamber, during the treatment. This method has disadvantages because, during the transfer from one chamber to the other, the plasmas have to be stopped. The system of airlocks requires the use and the management of vacuum manipulators, which complicates the equipment. Moreover, treating objects continuously cannot be carried out, at least not simply. Mention shall be made in particular, as documents representing this prior art, EP 2 630 271 or DE 10 2008 019427.

It is moreover known, from U.S. Pat. No. 6,296,735, a plasma treatment facility. The latter comprises an enclosure that can be placed in a vacuum, which encloses plasma chambers. In addition, a conveyor makes it possible to displace the objects between an upstream storage zone and a downstream storage zone. This facility does not make it possible to overcome the shortcomings that are inherent to the industrial solutions described hereinabove.

In addition, from US 2011/308456, a treatment facility is known provided with a barrel where the parts are arranged. The support of the barrel comprises in the center thereof an assembly allowing for the implementation of surface treatments. During each treatment, a vacuum is applied in the enclosure. This document does not provide a satisfactory solution, as it does not allow for a continuous supply of a high number of parts to be treated.

A method of plasma treatment and of sterilization is also known, that implements a conveyor of bottles made of plastic. This conveyor passes successively through a vacuum chamber, then in the treatment zone and finally in a second vacuum chamber. This method makes it possible to continuously treat bottles positioned in a line on the conveyor. This document does not however provide a satisfactory solution, as it implies supplying the parts one by one. Mention will be made in particular, as a document representing this prior art, of US 2008/2630271.

Finally, it is known, from U.S. Pat. No. 3,894,926, a device for transferring objects between a zone at atmospheric pressure and a zone intended to be placed under vacuum. This device comprises a frame, a housing arranged in this frame, as well as a transfer member, mobile in relation to the frame, which comprises cells for receiving objects to be transferred. The frame defines two openings in communication respectively with the zone at atmospheric pressure and the zone intended to be placed in a vacuum, as well as an intermediate region intended to be placed either under vacuum, or at atmospheric pressure. This transfer device cannot however be integrated into a treatment facility such as covered by the invention, without entailing significant technical difficulties.

An objective of this invention is to overcome, at least partially, the disadvantages of prior art mentioned hereinabove.

Another objective of the invention is to propose a vacuum treatment facility provided with an industrial device that makes it possible to transfer, to a treatment zone under vacuum, a large quantity of parts in a short time.

Another objective of the invention is to propose such a facility of which the transfer device authorizes a supply of the continuous type of these parts to this treatment zone under vacuum.

Another objective of the invention is to propose such a facility of which the transfer device has a relatively simple structure for the purpose of supplying and/or removing the parts to/from the zone under vacuum.

### OBJECT OF THE INVENTION

According to the invention, the objectives hereinabove are achieved by means of a surface treatment facility, comprising:

a main enclosure (1) under vacuum provided with treatment means, in particular of the plasma type,

at least one transfer device for transferring objects between a zone at atmospheric pressure (ZA) and a zone under vacuum (ZV), said at least one transfer device being provided upstream and/or downstream from the main enclosure and said at least one transfer device comprising:
a frame (51)
a housing (L) arranged in this frame,
a transfer member (55), movably mounted in the housing with respect to the frame, said transfer member comprising at least one cell (56-59) for receiving at least one object to be transferred,
said frame defining a first opening (52) in communication with the zone at atmospheric pressure, a second opening (53) in communication with the zone under vacuum, as well as an intermediate region (INT) placed either under vacuum, or at atmospheric pressure,
isolating means between the intermediate region and each opening,
driving means (60), adapted to drive each cell successively facing the first opening, the intermediate region, then the second opening,
   said facility further comprising:

means (30) for unstacking/stacking objects, placed facing the second opening of each transfer device,

means (11-14) for conveying objects,

elements (24, 25) for supporting each object, held by said conveying means.

According to other features of the treatment facility of the invention, taken separately or according to any technically compatible combination:
the transfer member is rotably mounted in the housing.
the isolating means are forming by a restriction in the section between facing, also called opposing, walls belonging respectively to the frame and to the transfer member.
the distance between the opposing walls, belonging respectively to the frame and to the transfer member, is less than 200 micrometers, in particular less than 100 micrometers, more particularly close to 50 micrometers.
at least one channel, associated with means of pumping, opens onto the intermediate region.
as a cross-sectional view, the transfer member comprises several cells, in particular four cells, angularly distributed in a regular manner.
as a front view, the transfer member comprises several cells longitudinally distributed in a regular manner.
the means for unstacking/stacking comprise two unstacking members placed one above the other, with each member mobile between a retaining position, wherein it opposes the falling of objects, and a releasing position, wherein they allow objects to fall via gravity.
each unstacking member comprises an elongated body carved with indentations, in particular semi-circular, distributed in a regular manner.
the means for conveying comprise at least two parallel conveyor belts, and several support elements are grouped together next to one another within a support member, extending transversally between at least two neighboring belts.
each support element comprises means for centering the object, distributed around a central opening.

The invention further has for object a method for implementing the facility hereinabove, wherein a first stack of objects is placed in a first cell opposite the zone at atmospheric pressure, the transfer member is displaced in such a way that the first cell is opposite, i.e. faces the intermediate region, the first cell opposite the intermediate region is placed in a vacuum, the transfer member is displaced in such a way that the first cell is opposite the zone under vacuum, the first stack of objects is deposited on the unstacking system, the objects of the first stack are unstacked, each unstacked object is deposited onto a respective support element.

According to other characteristics of the method of implementing the invention, taken separately or according to any technically compatible combination:
at the same time as the first cell opposite the intermediate region is placed in a vacuum, a second stack of objects is placed in a second cell opposite the zone at atmospheric pressure.
at the same time as the second cell opposite the intermediate region is placed in a vacuum, a third stack of objects is placed in a third cell opposite the zone at atmospheric pressure.
each object is deposited on the unstacking system via gravity.
the cell opposite the intermediate region is placed in a vacuum, for a duration between 10 and 60 seconds, preferably between 20 and 40 seconds.
the cell opposite the intermediate region is placed in a vacuum, at a pressure less than 5.10⁻² mbar, preferably at a pressure less than 10⁻² mbar.

The invention makes it possible to achieve the aforementioned objectives.

The transfer device used in the invention makes use of a zone that is constantly at atmospheric pressure, as well as a zone that is constantly under vacuum. In these conditions, only one intermediate region is to be subjected to being place in a vacuum periodically. Consequently, this operation of placing in a vacuum relates to a limited volume, in such a way that it can be carried out conveniently and quickly.

This transfer device therefore allows for a continuous supply of the treatment enclosure, placed downstream. A high number of objects can be admitted into this enclosure, via the device in accordance with the invention. Furthermore the means for unstacking/stacking objects within the chamber under vacuum allow for the temporary storage of objects, on hold to be transported by the means for conveying in the main enclosure.

In addition, this transfer device has a relatively simple mechanical structure. In particular, note that it makes use of a reduced number of parts in motion. In these conditions, the risks of malfunction are reduced, which prevents untimely stoppages of the vacuum treatment.

### DESCRIPTION OF THE FIGURES

The invention will be described hereinafter, in reference to the annexed drawings, provided solely as non-limiting examples, wherein:

FIGS. 1 and 2 are general views in perspective of a facility in accordance with the invention, respectively with and without a protective casing.

FIG. 3 is a perspective view, similar to FIG. 2, showing the facility in more detail in accordance with the invention.

FIG. 4a is a front view, showing the facility in accordance with the invention.

FIG. 4b is a top view, showing the bars of an unstacker belonging to the facility in accordance with the invention.

FIGS. 4c to 4f are front views and top views, diagrammatically showing the implementation of the unstacker belonging to the facility in accordance with the invention.

FIG. 5 is a perspective view, similar to FIG. 2, showing the means for conveying of the facility in more detail in accordance with the invention.

FIG. 6 is a cross-section view showing the interior of the transfer device belonging to the facility of the preceding figures.

FIGS. 7a to 7e are cross-section views, diagrammatically showing the implementation of the transfer device of FIG. 6.

The following numerical references are used in this description:

| | |
|---|---|
| 1 main enclosure | 50 interface chamber |
| 10 conveyor | 11-14 endless belt |
| 20 support | 30 unstacker |
| 19 spacer | 18 cylinder |
| 15 flat portion | 16 lug |
| 21 tab | 23 foot |
| 22 plate | 24 circular recess |
| 25 pin | 31, 32 unstacking bar |
| 34, 34′ elongated body | 35, 35′ wall |
| 51 frame | 52, 53 opening |
| 55 barrel | 54 horizontal channels |
| 56-59 cell | C1-C12 longitudinal cell |
| 60, 81, 82 motor | 80 transversal axis |
| 90, 90′ tip | CO1, CO2 collar |
| SR direction of rotation | SP direction of progression |
| ST direction of translation | ST1, ST2, ST3 translation |
| P1, P2, P3, P4 Part | F1, F2 rotation |

### DETAILED DESCRIPTION

The facility in accordance with the invention substantially comprises a main enclosure 1 under vacuum allowing for the surface treatment of parts, or objects, as well as an interface chamber 50. The latter, which forms a transfer device according to the invention, allows for the supply and the putting into a vacuum of these parts.

First of all, as shown in FIGS. 1 & 2, the main enclosure 1 comprises:
means for treating the surface of the parts. These means, of a type known per se, are not shown. This is in particular means of surface treatment of the plasma type;
a conveyor 10 comprised of four endless belts (11, 12, 13, 14) provided with drive means of the type known per se and not shown;
support members 20, comprised of several support elements of which the shape is adapted to that of the parts;
an unstacker 30 making it possible to unstack the parts to be treated from the chamber 50 then to place them on the supports 20.

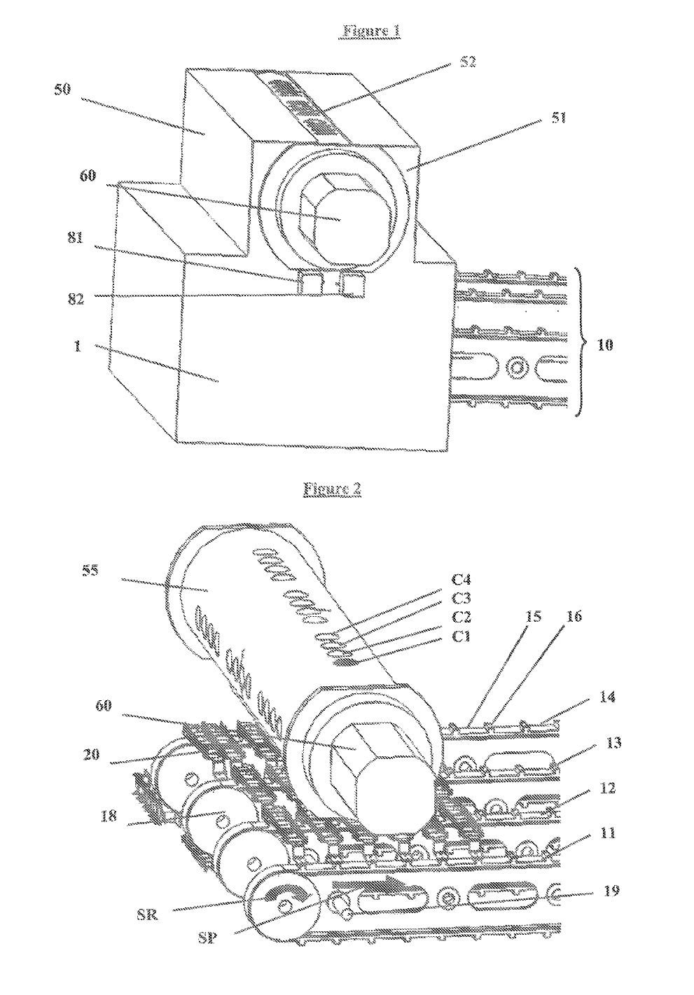

The conveyor 10, shown on FIGS. 2, 3 & 5, comprises four endless belts (11, 12, 13, 14) whereon are fixed the part supports 20. These belts are spaced apart from one another by an identical distance, by means of spacers 19. The endless belts (11, 12, 13, 14) are provided, at their ends, with a cylinder 18 allowing for the actuation thereof. The direction of rotation SR of these cylinders defines the direction of progression SP of the endless belt.

The shape and the dimensions of the endless belts (11, 12, 13, 14) are adapted to the supports 20 and to the objects to be treated. The belt (11, 12, 13, 14) comprises a flat portion 15 and lugs 16 arranged in a regular manner along the flat portion 15. These lugs 16 make it possible to securely fasten the part supports 20 onto the belts.

The conveyor 10 extends over the entire facility in accordance with the invention, from the upstream interface chamber 50 by passing through the plasma treatment zone or zones and to the downstream interface chamber. The latter, which is not shown, has for example a structure similar to the upstream chamber 50. The conveyor is included in the main chamber which is itself under vacuum.

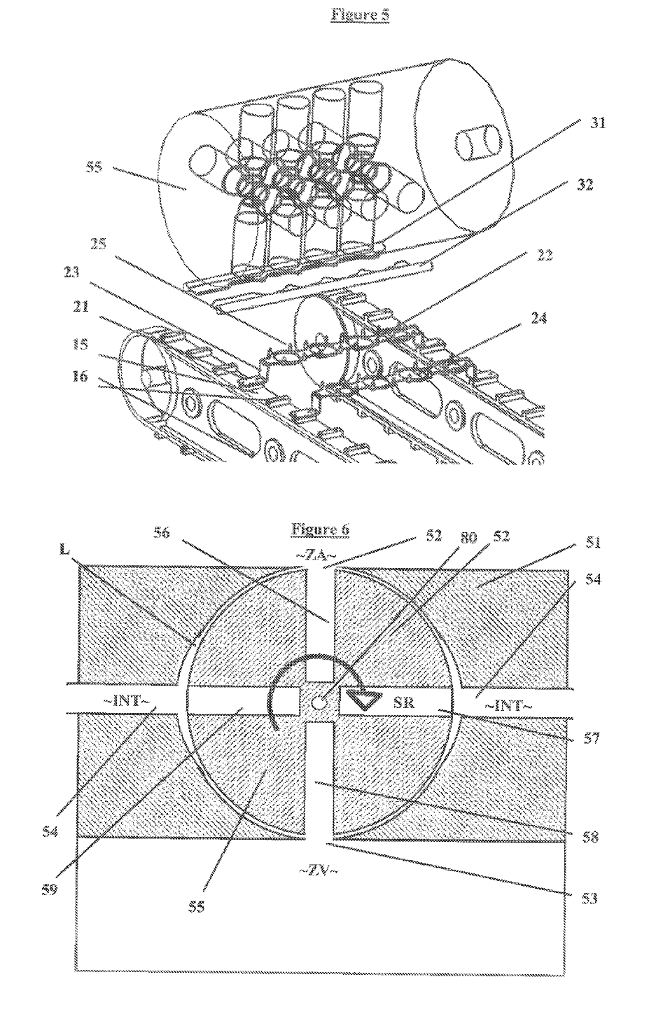

In the example shown in FIGS. 3 and 5, each support organ 20 transversally connects the endless belts (11, 12, 13, 14). This support organ, which has the shape of a bench, comprises several plates 22 of elongated shape, of which each one is prolonged at the two ends thereof by two feet 23. Each foot is furthermore terminated by a side tab 21 allowing for the fastening, by any suitable means, on a lug of an endless belt (11, 12, 13, 14).

Each plate 22, FIG. 5, is carved with four circular recesses 24 arranged in a regular manner over its length. Each circular recess 24 is bordered, at its periphery, by four diametrically opposite pins 25. These pins form support elements, intended to maintain and center the part to be treated. The recesses 24 provided on the plate 22 allow for an optimum surface treatment of the part, in particular in that they delimit passages that allow the plasma to reach, not only the outer face, but also the inner face of the parts to be treated.

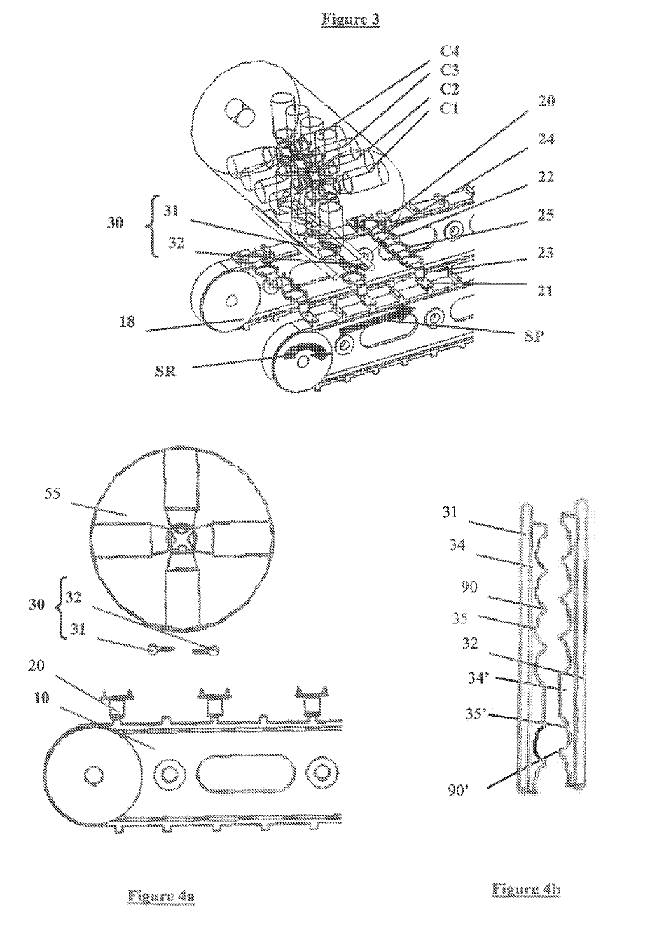

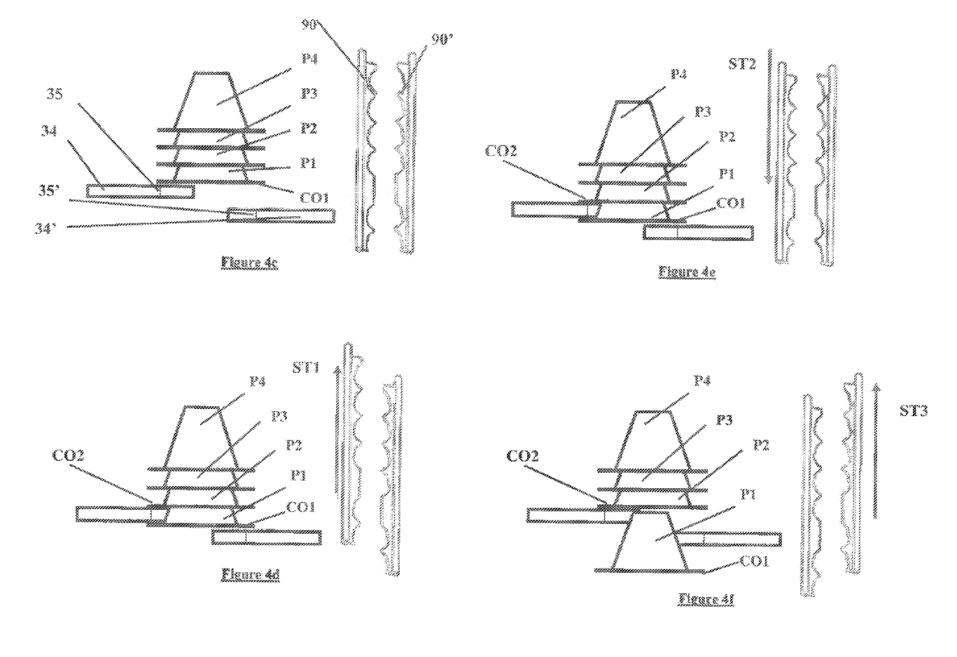

The station 30, FIGS. 4a and 4b, is intended for the unstacking of the objects to be treated. The shape and the dimensions of this unstacker 30 are adapted to the objects to be treated. In the example shown, this unstacker 30 first comprises two unstacking bars 31 and 32, mobile in translation according to a back-and-forth movement. The two bars are parallel and their separation is adapted to the dimensions of the parts to be treated. As shall be seen in what follows, the action of unstacking is possible by the positioning of the bar 31 above the bar 32. The two bars 31 and 32 have an identical structure and are arranged symmetrically, on either side of an axis parallel to the one of the supports 20. The bars include an elongated body 34 carved with semi-circular indentations, provided in a regular manner along the length.

The walls 35, bordering these indentations, are used to maintain the parts to be treated. The station 30 is located at a reduced distance from the conveyor, which allows for the unstacking by limiting the risk of breakage of parts. The distance between the unstacker 30 and the interface chamber 50 can confer the role of an intermediate reservoir, so as to not compromise the high treatment speed of the main enclosure. The unstacking devices 31 and 32 each include, at the end thereof, a respective drive motor 81 and 82, as can be seen in FIG. 1. The direction of displacement is defined by the arrow SP, in FIG. 2.

Then, the interface chamber 50 allowing for the putting into a vacuum comprises a frame 51 of elongated shape, a barrel 55 received in a housing L of this frame, as well as a vacuum pump, of a type known per se, which is not shown.

The shape and the dimensions of the frame 51 are adapted to the objects to be treated. In the example shown, FIG. 6, the frame 51 of rectangular shape allows for the introduction of the parts on the upper portion by the intermediary of an elongated so-called introduction opening 52. The latter opens onto the exterior, in such a way that it is in a zone at atmospheric pressure ZA. The lower portion of the frame 51 is carved with a removal opening 53, which opens onto the main enclosure which forms a zone under vacuum ZV.

The frame is furthermore carved with two horizontal channels 54, which delimit two intermediate zones INT, arranged between the introduction and removal openings. These channels are connected to a vacuum pump not shown that can be integrated into the frame or be exterior to the latter. The assembly of the pump with the channels 54 will define the aforementioned intermediate zone that can be, either at atmospheric pressure, or under vacuum. This function will appear clearly from the operating mode which shall be explained in what follows.

The frame comprises a transversal axis 80 that supports the barrel 55, which forms a transfer member in terms of the invention. The walls opposite the barrel and the frame define a functional clearance, which is between 20 and 200 micrometers, preferably between 50 and 100 micrometers, in particular about 50 micrometers.

This restriction in the section between the walls opposite the barrel and the frame forms isolating means, making it possible to limit the exchanges between, on the one hand, the intermediate regions and, on the other hand, the zone of atmospheric pressure or the zone under vacuum. In other terms, the putting respectively under vacuum or at atmospheric pressure of the intermediate regions is not substantially hindered by the existence of the zone respectively at atmospheric pressure or under vacuum. Alternatively to this restriction in section, or in addition to the latter, the isolating means can include seals placed on the walls of the frame and/or of the barrel.

The shape and the dimensions of the barrel 55 are adapted to the objects to be treated. In the example shown, the barrel 55 is a cylinder comprising, as a cross-section, four cells 56, 57, 58, 59 angularly distributed in a regular manner, at the periphery of the barrel 55. Moreover the barrel 55 comprises several cells according to its longitudinal dimension, here twelve cells referenced from C1 to C12. The barrel 55 comprises a drive motor 60, at the end thereof. The direction of rotation is defined by the arrow SR.

We shall now describe the implementation of the facility, described hereinabove in reference to FIGS. 1 to 6.

Parts that have a truncated veil of which the first end is closed with a bottom and of which the second open end is radially extended by a collar are used. Typically, this part is therefore for example a goblet.

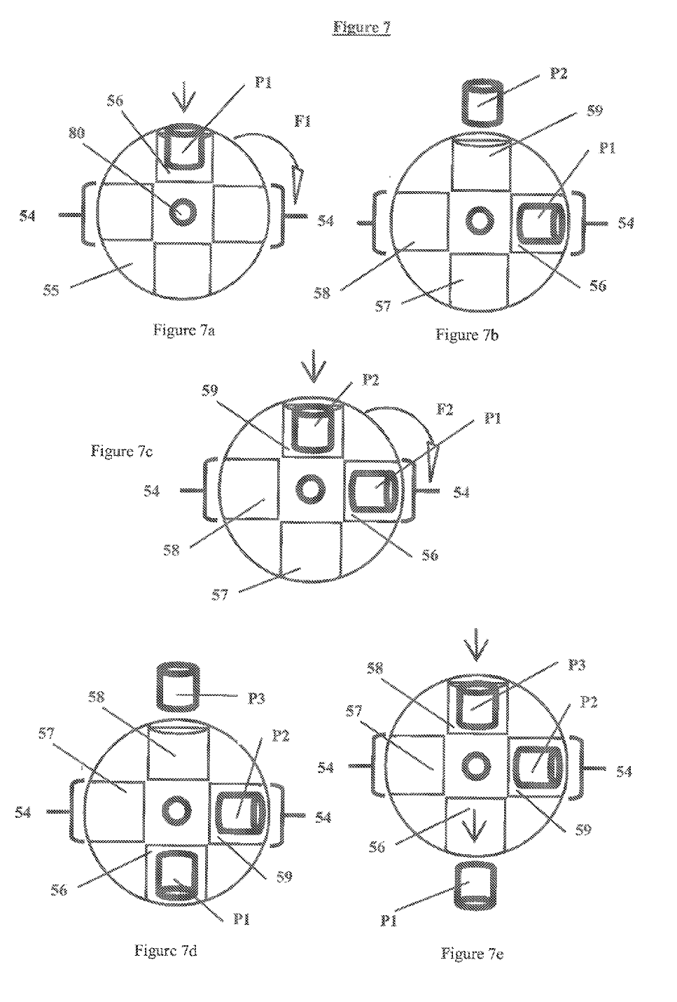

The implementation of the transfer device is described in FIGS. 7a to 7e, in reference to a single cell, in the longitudinal direction. However, this implementation is simultaneous for all of the goblets placed longitudinally one behind the other, in the cells C1 to C12.

The method comprises the following steps of implementation:

In the step 1 FIG. 7a, the cell 56 of the barrel 55 receives a set, or stack, of parts P1 through the opening 52 of the frame 51. This stack, which can include one to several dozen parts, is located in a zone at atmospheric pressure. After a duration of which the value is explained in what follows, the barrel 55 is actuated in rotation according to the axis central 80 by one quarter turn, according to arrow F1.

In the step 2, FIG. 7b, the cell 56 arrives in the intermediate region facing the channel 54. The set of parts P1, which is then opposite the region INT, passes from an atmospheric pressure state to a vacuum state, FIG. 7c. During this putting into a vacuum, the interior volume of the cell is placed at a pressure less than 5.10⁻² mbar, preferably at a pressure less than 10⁻² mbar.

Note that, advantageously, the vacuum pump associated with the channel 54 is activated continuously. When no cell is placed opposite the channel, the pump exerts a suction opposite a solid portion, namely the body of the barrel, which has no significant effect. At the same time, the adjacent cell 59 is loaded with another stack of parts P2. After a duration of which the value is explained in what follows, the barrel 55 is actuated in rotation by a quarter turn, according to arrow F2.

In the step 3, FIG. 7d, the cell 56 positions itself vertically facing the unstacker 30, the cell 59 is positioned facing the channel 54. As shown in FIG. 7e, the parts of the first stack P1 fall by gravity towards the station 30, according to arrow G. Simultaneously, the stack of parts P2 passes from an atmospheric pressure state to a vacuum state, as described hereinabove for the stack P3, while the following cell 58 is loaded with a third stack of parts P3. After a duration of which the value is explained in what follows, the barrel 55 is actuated in rotation by one quarter turn.

Then the stack of parts P2 falls by gravity, from the cell 59 to the unstacking station 30. Simultaneously, the parts P3 of the cell 58 pass from an atmospheric pressure state to a vacuum state, while the cell 57 is loaded with an additional stack of parts, not shown. The cell 56 of the barrel 55 then returns to its starting point, namely in top position.

The cycle of the actions on different cells 56 to 59 is therefore identical, being shifted in terms of time. Note that it is advantageous to provide four cells, because this allows for an introduction of the parts at atmospheric pressure, as well as a removal of the parts under vacuum, both carried out by gravity.

The transfer and action times on the cells (56, 57, 58, 59) are defined as follows:
the rotation/transfer time is typically within a range from two to six seconds;
the working time on a cell depends on the volume of the cell. The greater this volume is, the greater the pumping time for putting the latter in a vacuum will be. In the example described hereinabove, the time required is typically about thirty seconds in order to carry out the passage from atmospheric pressure to the vacuum.

The unstacker 30 allows for the supply of the support elements driven by the four endless belts (11, 12, 13, 14). The method of operation thereof is as follows.

It is supposed that a stack of parts P1 to P4 has just fallen by gravity from one of the cells of the transfer member 50, see FIG. 4c. The collar CO1 of the lower part P1 is then bearing against a tip 90 of an indentation 35, arranged in the upper unstacking bar 31. This bar 31 is in its initial position, wherein it retains the parts, namely it opposes the falling thereof by gravity.

Then, as shown FIG. 4d, the bar 31 is placed in translation according to arrow ST1, in such a way as to displace it from its initial retaining position hereinabove, to a so-called retracted or release position. The stack P1-P4 then falls by gravity, and the collar CO1 of the first part P1 abuts against a tip 90′ of an indentation 35′ of the lower unstacking bar 32. The latter is in its initial retaining position, in the terms defined immediately hereinabove.

Then, see FIG. 4e, the upper unstacking bar repositions itself in its initial retaining position, according to arrow ST2, between the collar CO1 of the first part P1 and that CO2 of the second part P2. Then, see FIG. 4f, the lower unstacking bar 32 is then displaced according to arrow ST3 to its retracted release position, which allows the part P1 to fall by gravity to a support 20. Then the lower unstacking bar 32 returns to its initial retaining position, an operation that is not shown in the figures.

The sequence is repeated indefinitely. The unstacking operates in a synchronized manner with the direction of progression SR of the four endless belts (11, 12, 13), so as to deposit the parts on the support members 20.

As mentioned hereinabove, the unstacker is used as an intermediate reservoir of parts. When the cell 56 is located above the unstacker 30 and is depositing the parts, the pumping action operating on the following cell 57, makes it possible to unstack the excess parts limiting the rotation of the barrel 55. There are then a few parts left on the unstacker allowing for the supply of parts on the supports, until the cell 57 after rotation of the barrel again supplies the unstacker.

In the example hereinabove, the transfer chamber 50 is located upstream in the facility. However a downstream transfer chamber can be provided allowing for the extraction of the parts treated as such, outside of the main enclosure 1. In this case, this chamber is placed “head at the bottom” in relation to that shown in particular in FIG. 6. The treated parts fall for example by gravity into a cell of the barrel, placed in the zone under vacuum. Then the barrel is placed in rotation, in such a way that these parts are put back at atmospheric pressure in the intermediate region. Finally these parts are removed from this cell, typically by gravity, to the zone at atmospheric pressure.

1. Surface treatment facility, comprising:
a main enclosure (1) under vacuum provided with treatment means, in particular of the plasma type,
at least one transfer device for transferring objects between a zone at atmospheric pressure (ZA) and a zone under vacuum (ZV), said at least one transfer device being provided upstream and/or downstream from the main enclosure and said at least one transfer device comprising:
a frame (51)
a housing (L) arranged in this frame,
a transfer member (55), movably mounted in the housing with respect to the frame, said transfer member comprising at least one cell (56-59) for receiving at least one object to be transferred,
said frame defining a first opening (52) in communication with the zone at atmospheric pressure, a second opening (53) in communication with the zone under vacuum, as well as an intermediate region (INT) placed either under vacuum, or at atmospheric pressure,
isolating means between the intermediate region and each opening,
driving means (60), adapted to drive each cell successively facing the first opening, the intermediate region, then the second opening,
said facility further comprising:
means (30) for unstacking/stacking objects, placed facing the second opening of each transfer device,
means (11-14) for conveying objects,
elements (24, 25) for supporting each object, held by said conveying means.

2. Surface treatment facility according to claim 1, wherein the transfer member (55) is rotably mounted in the housing (L).

3. Surface treatment facility according to claim 1, wherein the isolating means are forming by a restriction in the section between facing walls belonging respectively to the frame (51) and to the transfer member (55).

4. Surface treatment facility according to claim 1, wherein at least one channel (54), associated with means of pumping, opens onto the intermediate region.

5. Surface treatment facility according to claim 1, wherein, as a cross-sectional view, the transfer member (55) comprises several cells (56-59), in particular four cells, angularly distributed in a regular manner.

6. Surface treatment facility according to claim 1, wherein, as a front view, the transfer member (55) comprises several cells (C1-C12), longitudinally distributed in a regular manner.

7. Surface treatment facility according to claim 1, wherein the distance between the facing walls, belonging respectively to the frame and to the transfer member, is less than 200 micrometers, in particular less than 100 micrometers, more particularly close to 50 micrometers.

8. Surface treatment facility according to claim 1, wherein in that the means for conveying comprise at least two parallel conveyor belts (11-14), and several support elements (24, 25) are grouped together next to one another within a support member (20), extending transversally between at least two neighboring belts.

9. Surface treatment facility according to claim 1, wherein the means for unstacking/stacking comprise two unstacking members (31, 32) placed one above the other, each member being mobile between a retaining position, wherein it opposes the falling of objects, and a releasing position, wherein they allow objects to fall via gravity.

10. Surface treatment facility according to claim 1, wherein each unstacking member comprises an elongated body (34) carved with indentations, semi-circular in particular, distributed in a regular manner.

11. Method for implementing the facility according to claim 1, wherein:
a first stack of objects (P1) is placed in a first cell (56) facing the zone at atmospheric pressure (ZA),
the transfer member is displaced in such a way that the first cell faces the intermediate region (INT),
the first cell facing the intermediate region is placed under vacuum,
the transfer member is displaced in such a way that the first cell faces the zone under vacuum (ZV),
the first stack of objects is deposited on the unstacking system,
the objects of the first stack are unstacked,
each unstacked object is deposited onto a respective support element.

12. Method of claim 11, wherein the cell facing the intermediate region is placed under vacuum, at a pressure less than 5.10⁻² mbar, preferably at a pressure less than 10⁻² mbar.

13. Method of claim 11, wherein, at the same time as the first cell facing the intermediate region is placed under vacuum, a second stack of objects is placed in a second cell facing the zone at atmospheric pressure.

14. Method of claim 13, wherein, at the same time as the second cell facing the intermediate region is placed under vacuum, a third stack of objects is placed in a third cell facing the zone at atmospheric pressure.

15. Method of claim 11, wherein the cell facing the intermediate region is placed under vacuum, for a duration between 10 and 60 seconds, preferably between 20 and 40 seconds.
